(19) United States
(12) Patent Application Publication
(10) Pub. No.: US 2014/0011277 A1
(43) Pub. Date: Jan. 9, 2014

(54) NOVEL THERAPEUTIC STRATEGIES FOR IMPROVING AN ANTICANCER TREATMENT

(71) Applicants: Isabelle Mus-Veteau, Grasse (FR); Michel Bidet, Saint-Laurent-Du-Var (FR)

(72) Inventors: Isabelle Mus-Veteau, Grasse (FR); Michel Bidet, Saint-Laurent-Du-Var (FR)

(73) Assignee: CENTRE NATIONAL DE LA RECHERCHE SCIENTIFIQUE (CNRS), Paris (FR)

(21) Appl. No.: 13/993,934

(22) PCT Filed: Dec. 9, 2011
(86) PCT No.: PCT/FR11/52927

§ 371 Date: Sep. 26, 2013

(30) Foreign Application Priority Data
Dec. 13, 2010 (FR) 1004840

(51) Int. Cl. C12N 5/09

(52) U.S. Cl. CPC C12N 5/0693
USPC 435/375

(57) ABSTRACT

The subject of the invention is novel medicaments intended to modulate the intracellular concentration of anticancer agents through the modulation of the activity of Patched protein.

The invention is used in particular in the improvement of the treatment of cancers.

The Hedgehog (Hh) signalling pathway plays an important role in growth and structuring during the course of embryonic development.

The Hh signalling pathway in mammals makes use of a large number of effectors, including at least
the proteins of the Hh family encoded by three distinct genes Shh (Sonic Hedgehog (Shh), Indian Hedgehog (Ihh) and Desert Hedgehog (Dhh),
the proteins responsible for the secretion and transport of the Hh proteins, including Dispatched (Disp),
Shifted, which acts at long range and is required for the accumulation of the Hh proteins,
the receptor proteins of the Hh signal: Patched (Ptc), Hedgehog interacting protein (Hip);
Smoothened (Smo) responsible for the transmission o the Hh signal within the receptor cell.

It is generally accepted in the prior art that Smo is constitutively inhibited by Ptc in the absence of Hh proteins and that the binding of the Hh ligand to its receptor Ptc results in the activation of Smo which in turn will activate the zinc finger transcription factors, the Gli factors.

It is well documented that disorders of the Hh signalling pathway induce congenital malformations and disorders such as Gorlin syndrome and holopros-encephaly in humans.

Likewise, it is known that individuals carrying a defective allele of the gene PTCH1, leading to a reduction in the activity of the corresponding protein, exhibit a set of morphological defects, particularly in the fingers, face, teeth and ribs, grouped under the name of Gorlin syndrome.

The Patched protein is a membrane protein which crosses the membrane 12 times and interacts with the Hh protein in the two large extracellular loops.

The homologies of its sequence with those of the bacterial transporters of the RND (resistance, nodulation, division) family suggest that this protein could have a transporter function.

However, to the knowledge of the applicant, the transporter role of Patched protein, in particular that of transporter of anticancer agents, has never been demonstrated.

Surprisingly, and after prolonged studies and using doxorubicin as a prototype of intracellular anticancer agents, the applicant has been able to demonstrate by several approaches on different cellular models (see the examples) that Patched protein is capable of binding intracellular anticancer agents, particularly doxorubicin, and that Patched protein could be responsible for the efflux of intracellular anticancer agents, in particular doxorubicin.

By efflux of intracellular anticancer agents and/or of doxorubicin, is here meant that under the influence of Patched protein the intracellular anticancer agents and/or doxorubicin can be transported to the outside of the cell.

In the absence of the Hh protein, the membrane protein Patched is present in the plasma membrane of the cells, and the intracellular concentration of anticancer agents, in particular doxorubicin, decreases with the increase in the efflux of the anticancer agents, in particular doxorubicin, induced by Patched.

Conversely, when the Hh protein is secreted into the extracellular medium, it interacts with Patched, and the complex Patched/Hh is then internalized and degraded. A consequence of the disappearance of Patched protein in the plasma membrane is the cessation of the efflux of anticancer agents, in particular doxorubicin, and thus an increase in the intracellular concentration of anticancer agents, in particular doxorubicin.

Thus the various results obtained by the applicant lead them to propose that Patched protein could be a transmembrane transporter and could be involved in the efflux of anticancer agents, in particular doxorubicin.

Thus regulation of the activity of Patched protein could make it possible to regulate the intracellular concentration of anticancer agents, in particular doxorubicin.

The discovery of this novel activity of Patched protein makes it possible to envisage novel ways of improving the efficacy of anticancer treatments through the regulation of the intracellular concentration of anticancer agents, in particular doxorubicin.

Thus a first subject of the invention is an in vitro method for regulation of the intracellular concentration of intracellular anticancer agents characterized in that cells and a modulator of the activity of Patched protein are placed in contact in order to regulate the intracellular concentration of anticancer agents.

A person skilled in the art is well aware that during treatments by means of anticancer agents, in particular those with an intracellular target, the various protocols recommend the use either of a single anticancer agent or of combinations of several anticancer agents. In the present text also, the use of the expression “anticancer agents” refers to one as well as to several intracellular anticancer agents.

By modulator is here meant an activator as well as an inhibitor.

Thus according to a variant of the invention the method is an in vitro method for increasing the intracellular concentration of anticancer agent, characterized in that cells and an inhibitor of the activity of transport of anticancer agents by Patched protein are placed in contact.

And according to another variant of the invention the method is an in vitro method for decreasing the intracellular concentration of anticancer agents, characterized in that cells and a stimulator of the activity of transport of anticancer agents by Patched protein are placed in contact.

Therapeutic applications can also be envisaged.

A subject of the invention is thus also the use of a modulator of the activity of Patched protein for the preparation of a medicament intended to regulate the intracellular concentration of anticancer agents.

According to a variant, a subject of the invention is the use of an activator of the activity of Patched protein for the preparation of a medicament intended to decrease the intracellular concentration of anticancer agents.

And according to another variant, a subject of the invention is the use of an inhibitor of the activity of Patched protein for the preparation of a medicament intended to increase the intracellular concentration of anticancer agents.

According to the invention, by inhibitor of Patched protein is meant:
antibodies directed against Patched protein as is shown by the study by Nakamura Masafumi et al., Anticancer Research 27, 3743-3748 (2007)
cholesterol, oxysterols, or other analogues of cholesterol. In fact, the inventors have shown (international application No. PCT/FR2011/000558, filed 18/10/2011 and Bidet et al., Plos One, September 2011, vol. 6, Issue 9, 2011) that Patched protein transports cholesterol from the inside to the outside of the cell. It is probable that the presence of a sufficient concentration of cholesterol or of one of its analogues creates competition with the anticancer agent on the Patched protein and inhibits the exit of the anticancer agent.
non-transported analogues of anticancer agents.

According to the invention, by anticancer agents is meant antitumour antibiotics, alkylating agents, antimetabolites, plant alkaloids, topoisomerase inhibitors or also mitotic spindle poisons.

As antitumour antibiotics, for example bleomycin, daunorubicin, doxorubicin, epirubicin, hydroxyurea, idarubicin, mitomycin C or also mitoxanthrone may be mentioned;

As alkylating agents, for example busulfan, carboplatin, chlorambucil, cisplatin, cyclophosphamide, ifosfamide, melphalan, mechlorethamine, oxaliplatin, uramustin or also temozolomide may be mentioned;

As antimetabolites, for example azathioprine, capecitabine, cytarabine, floxuridine, fludarabine, fluorouracil, gemcitabine, methotrexate or also pemetrexed may be mentioned;

As plant alkaloids, for example vinblastine or also vincristine (vinorelbine) may be mentioned;

As topoisomerase inhibitors, for example irinotecan, topotecan or also etoposide may be mentioned;

And finally, as mitotic spindle poisons, for example docetaxel, paclitaxel, vinblastine, vincristine or also vinorelbine may be mentioned.

Advantageously, according to the invention, the intracellular anticancer agent (or agents) particularly targeted can belong to the family of the antitumour antibiotics and still more advantageously the intracellular anticancer agent particularly targeted is doxorubicin.

According to the invention, the modulator (activator or inhibitor) of the activity of Patched protein is advantageously present in the medicament at physiologically effective doses.

According to the invention, the said medicament can be formulated for the digestive or parenteral route.

The medicaments according to the invention can further contain, in combination with a modulator of the activity of Patched protein, at least one other therapeutically active ingredient, whether it is active against the same pathology or against a different pathology, for use simultaneously, separately or staggered over time, in particular during a treatment in a subject affected by one of the aforementioned pathologies. As an example of another active ingredient a statin, an antagonist of the receptor Smoothened or also another modulator of the activity of efflux of an anticancer agent, different from a modulator of the activity of Patched protein, can be mentioned. A subject of the invention is thus also the combined use of a modulator of the activity of Patched protein, with a statin, and/or an antagonist of the receptor Smoothened and/or also another modulator of the activity of efflux of an anticancer agent, different from a modulator of the activity of Patched protein. Preferably, the modulator of the activity of Patched protein can be an inhibitor of the activity of Patched protein.

According to the invention, the modulator (activator or inhibitor) of the activity of Patched protein can be used in the medicament, mixed with one or more excipients or inert, i.e. pharmaceutically inactive and non-toxic, vehicles.

For example, saline, physiological, isotonic, buffered, etc., solutions compatible with a pharmaceutical use and known to those skilled in the art can be mentioned. The compositions can contain one or more agents or vehicles selected from dispersants, solubilisers, stabilisers, preservatives, etc.

Agents or vehicles which can be used in (liquid and/or injectable and/or solid) formulations are in particular methylcellulose, hydroxymethylcellulose, carboxymethylcellulose, cyclodextrins, polysorbate 80, mannitol, gelatine, lactose, plant or animal oils, acacia etc. Preferably, plant oils are used. The compositions can be formulated in the form of injectable suspension, gels, oils, tablets, suppositories, powders, gel capsules, capsules, etc., optionally by means of galenical forms or devices ensuring prolonged and/or delayed release. For this type of formulation, an agent such as cellulose, carbonates or starches is advantageously used.

Administration can be carried out by any method known to a person skilled in the art, preferably by the oral route or by injection, typically by the intraperitoneal, intra-cerebral, intrathecal, intravenous, intra-arterial or intramuscular route. Administration by the oral route is preferred.

The invention can be used in mammals, in particular in humans.

In general, the daily dose of the compound will be the minimum dose for obtaining the desired therapeutic effect. If necessary, the daily dose can be administered in two, three, four, five, six or more intakes per day or through multiple sub-doses administered at appropriate intervals during the day.

The quantity selected will depend on multiple factors, in particular on the route of administration, the duration of administration, the time of administration, the rate of elimination of the modulator (activator or inhibitor) of the activity of Patched protein, the product or various products used in combination with the modulator (activator or inhibitor) of the activity of Patched protein, the age, weight and physical condition of the patient, and also on his or her medical history and on any other information known in medicine.

A subject of the invention is also the use of a modulator of the activity of Patched protein in an anticancer treatment, advantageously for improving the efficacy of an anticancer treatment.

Other characteristics and advantages of the invention will become apparent on reading the embodiment examples of the invention that follow, and on examination of the figures, among which

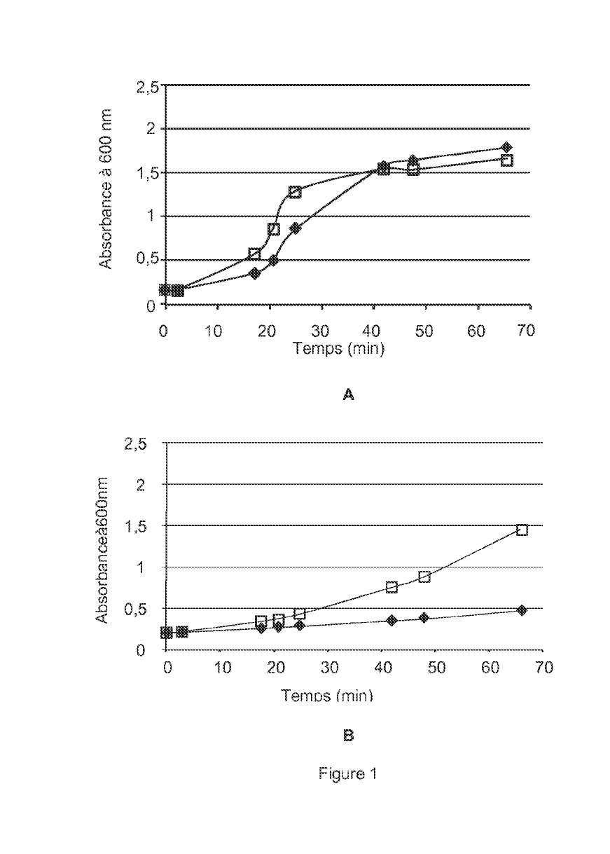

FIG. 1 shows the effect of doxorubicin on the resistance of yeasts expressing human Patched protein (□) or not expressing it (K699) ( [symbol] )

1A: in cultures in the absence of doxorubicin;

1B: in cultures in the presence of 10 μg/mL of doxorubicin.

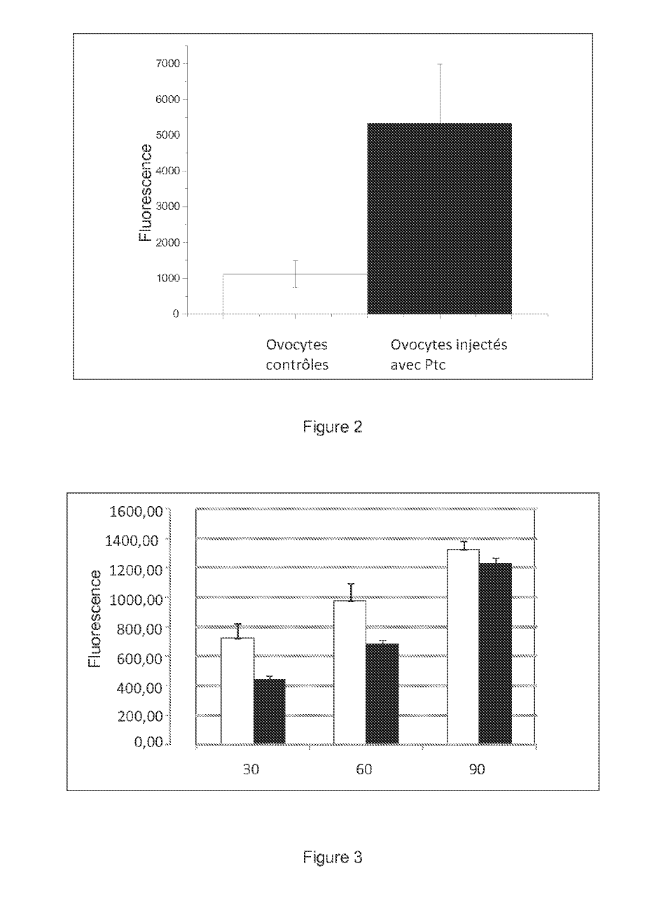

FIG. 2 shows the results of the study of the efflux of doxorubicin in yeasts expressing Patched protein (Ptc) or not expressing it (K699). The fluorescence values are given in arbitrary units.

FIG. 3 shows the results of experiments carried out in order to determine whether or not the endogenous protein Patched of the fibroblasts of NIH 3T3 mice promotes the efflux of doxorubicin. The fluorescence values are given in arbitrary units, (□) Control without Shh, and ( [symbol] ) with Shh

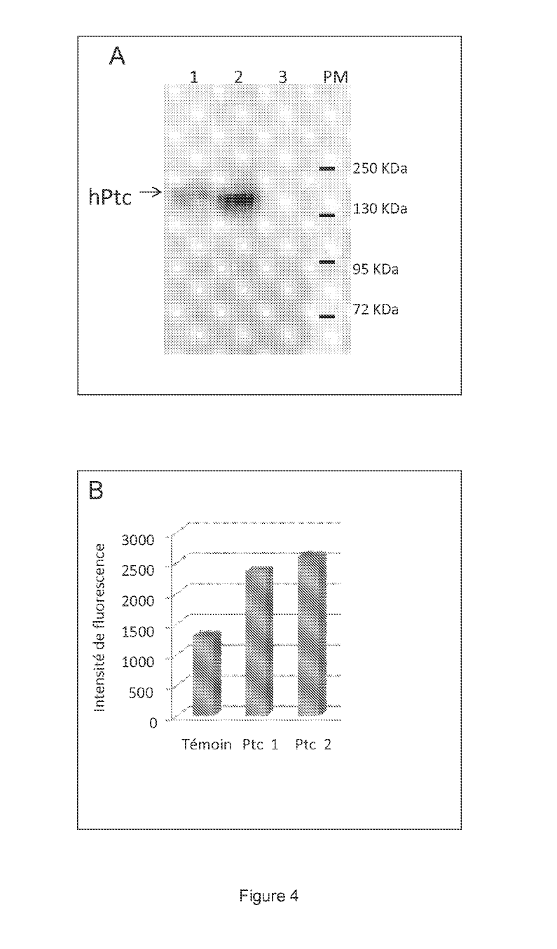

FIG. 4 shows the results of experiments carried out on xenopus oocytes and showing the influence of Patched protein on the efflux of doxorubicin.

4A: Western Blot on preparations of membranes of oocytes injected with different quantities of ribonucleic acid encoding human Patched protein (RNA of human Patched protein).

4B: Measurement of the efflux of doxorubicin in xenopus oocytes injected with various quantities of human Patched protein RNA. The fluorescence values are given in arbitrary units.

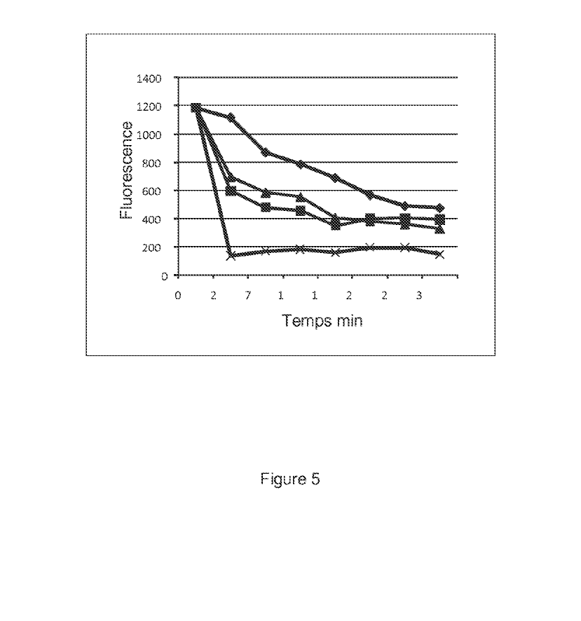

FIG. 5 shows the results of the study of the binding of doxorubicin to the Patched protein present in yeasts expressing or not expressing Patched protein.

### EXAMPLES

### Materials and Methods

### Cell Culture and Preparation of Membranes

The NIH 3T3 mouse fibroblast cell line was maintained in a DMEM culture medium (Invitrogen, USA) supplemented with 10% of foetal calf serum, 100 U/ml of penicillin and 100 μg/mL of streptomycin at 37° C. in a 5% CO₂ atmosphere saturated with water.

The cells were cultured in monolayer to 90-100% confluence and subjected to not more than 20 cell passages with subculturing every 4/5 days.

### Yeast Culture and Preparation of Membranes

The K699 strain of S. cerevisiae (Mata, ura3, and leu 2-3) was transformed with the plasmid YEpPMAhPtc-MAP (Joubert et al., 2009, BBA Biomembrane, 1788: 1813-1821; 2010, Methods in Molecular Biology, Humana Press, Mus-Veteau (Ed.), 601: 87-103) by the lithium acetate procedure and plated in culture dishes containing a minimal medium and a mixture of amino acids without leucine.

The clones were pre-cultured at 30° C. up to an optical density of 3 at 600 nm (OD₆₀₀) on a minimal medium (MM) (0.67% of yeast nitrogen base without amino acids, 0.3 mM adenine, 0.5 mM uracil, 0.3 mM tyrosine and a mixture of amino acids free of leucine) supplemented with 2% of D-glucose.

This preculture is then diluted to an OD₆₀₀ of 0.1-0.2 in a rich medium (yeast extract, bactopeptone, adenine) containing 2% of D-glucose and cultured at 18° C. with stirring at 200 rpm up to an OD₆₀₀ of 5-7.

For the preparation of the membranes, all the measurements were carried out at 4° C.

The yeasts were washed with cold water, resuspended in 50 mM Tris-HCl (pH 7.4), 150 mM NaCl, 2.5 mM EDTA, 1 mM PMSF and 4 mM benzarmidine, and disrupted twice by vortex stirring for 15 mins in the presence of glass beads (425 to 600 μm, Sigma).

The non-ruptured yeasts and the glass beads were removed by centrifugation for 5 mins at 2000 g.

The membranes were collected by centrifugation of the obtained supernatant for 1 hour at 100,000 g.

The pellet was washed twice in the same buffer without EDTA and resuspended at 5 mg/ml in a buffer containing 50 mM Tris-HCl (pH 7.4), 150 mM NaCl, 10% of glycerol, 1 mM PMSF and 4 mM benzamidine.

### Gel Electrophoresis and Western Blot

The proteins contained in the samples were separated by electrophoresis on 8% SDS/polyacrylamide gel (8% SDS-PAGE) and transferred onto a nitrocellulose membrane (Amersham) using standard techniques.

The nitrocellulose membranes were firstly blocked for 1 hour at ambient temperature in a blocking buffer (20 mM Tris-HCl (pH 7.5), 450 mM NaCl, 0.1% Tween-20, 4% skimmed milk), then placed in contact overnight at 4° C. with rabbit anti-PTC polyclonal antibodies (dilution 1:1000) (Joubert et al. 2009).

The nitrocellulose membranes were then washed twice in the blocking buffer.

Goat anti-rabbit secondary polyclonal antibodies (dilution 1:3000) coupled with horseradish peroxidase (Dako) were then applied for 2 hrs at 4° C. The detection of the reactions was carried out using the ECL kit (Millipore), and a Fusion FX7® (Vilber-Lourmat) chemiluminescence detection system for the Western blot.

### Resistance of Yeasts to Doxorubicin

Yeasts not expressing human Patched protein (controls) K699 and yeasts expressing human Patched protein were cultured at 18° C. in normal rich medium and in rich medium with 10 μg of added doxorubicin per mL of medium.

The OD of the yeasts at 600 nm is measured over time.

### Measurement of the Efflux of Doxorubicin

on NIH 3T3 cells: for the doxorubicin efflux experiments, NIH 313 cells were seeded into the wells of 24-well plates in DMEM culture medium (Invitrogen, USA) supplemented with 10% of foetal calf serum, 100 U/ml of penicillin and 100 μg/mL of streptomycin at 37° C. in a 5% CO₂ atmosphere saturated with water. At 80% confluence, the medium is removed and replaced by medium containing 10 μM of doxorubicin. The cells are then incubated for 2 hrs at 37° C. with stirring at 50 rpm. The cells are then rinsed with NaCl buffer (140 mM NaCl, 5 mM KCl, 1 mM CaCl₂, 1 mM MgSO₄, 5 mM glucose, 20 mM HEPES, pH 7.4), then incubated with 250 μL of NaCl buffer (140 mM NaCl, 5 mM KCl, 1 mM CaCl₂, 1 mM MgSO₄, 5 mM glucose, 20 mM HEPES, pH 7.4) in the absence or presence of 30 nM of Shh for 30, 60 and 90 minutes.

After a centrifugation (5 mins at 6800 g) to remove all cell fragments, the reading of the doxorubicin fluorescence (λex=490+/−10 nm, λem=610+/−10 nm) was carried out on 200 μL of supernatant in a plate reader (FLUOstar, Labtech).
on yeasts: K699 control yeasts and yeasts expressing human Patched protein were cultured at 18° C. up to an OD₆₀₀ of 7 on a rich medium with 2% of added D-glucose.

The cells are then centrifuged, (5 mins. 3000 g), washed 4 times with water and finally resuspended carefully to an OD₆₀₀ of 10 in 50 mM NaOH-Hepes buffer (pH 7.0) and incubated for 2 hours with 10 μM of doxorubicin.

After rinsing, the yeasts are resuspended in 50 mM NaOH-Hepes buffer (pH 7.0).

After 20 mins, the yeasts are centrifuged and doxorubicin contained in the supernatant is measured by fluorescence (λex=490+/−10 nm, λem=610+/−10 nm).
On xenopus oocytes: obtaining and preparing the oocytes

The oocytes are collected by surgery on an Xenopus laevis female anaesthetized with 0.2% of MS222 (tricaine methanesulfonate) in accordance with the procedures recommended by the ethics committee. The oocytes are washed with an MBS saline solution, pH 7.4 (NaCl 85 mM, KCl 1 mM, NaHCO₃2,4 mM, MgSO₄0.82 mM, Ca(NO₃)₂0.33 mM, CaCl₂ 0.41 mM, HEPES 10 mM, NaOH 4.5 mM) supplemented with 50 U/ml of penicillin and 50 μg/ml of streptomycin. The oocytes are then defolliculated by incubation for 16 hrs at 19° C. with 1.8 mg/ml of collagenase then 30 mins with MBS without calcium, and placed in MBS. 50 nL containing 10 or 20 ng of RNA encoding human Patched protein are injected per oocyte. The oocytes are incubated for 3 days at 19° C. in MBS. 4 lots of 10 control oocytes (not injected) and oocytes injected with RNA encoding human Patched protein were incubated for 16 hrs at 19° C. with 100 μM of doxorubicin. After rinsing, the oocytes are placed in MBS. The supernatant is sampled after 90 mins and the doxorubicin fluorescence present in the sample is measured by fluorescence (λex=490+/−10 nm, λem=610+/−10 nm).

### Binding of Doxorubicin to Patched Protein

The binding of doxorubicin to Patched protein is demonstrated by quenching of doxorubicin fluorescence on membrane preparations from K699 control yeasts and from yeasts expressing human Patched protein.

50 nM of doxorubicin are added to 30 μg of membranes and the doxorubicin fluorescence is measured as a function of time.

### EXAMPLE 1

### Yeasts Expressing Human Patched Protein Resist Doxorubicin Better than Yeasts not Expressing it (FIG. 1)

In normal rich medium (1A), the K699 control yeasts (▪) and the yeasts expressing human Patched protein (♦) exhibit similar growth over time.

In the presence of doxorubicin (1B), the yeasts expressing human Patched protein resist better than the control yeasts.

### EXAMPLE 2

### Human Patched Protein Expressed in Yeast Promotes the Efflux of Doxorubicin (FIG. 2)

It is observed that the presence of human Patched protein on the membrane of yeasts causes an increase in the efflux of doxorubicin.

### EXAMPLE 3

### Endogenous Patched Protein Promotes the Efflux of Doxorubicin in Mouse Fibroblasts (FIG. 3)

In order to check whether Patched protein has the same function in the endogenous system, NIH 3T3 mouse fibroblasts were cultured to confluence in 24-well plates, then incubated in the presence of doxorubicin.

It is observed that the presence of Shh inhibits the efflux of doxorubicin by 20 to 30%. Shh interacts with the Patched protein on the surface of the cells and causes its internalization.

The decrease in the efflux of doxorubicin observed in presence of Shh could thus be due to the disappearance of Patched protein from the surface of the cell.

### EXAMPLE 4

### Human Patched Protein Expressed in Xenopus Oocytes Promotes the Efflux of Doxorubicin (FIG. 4)

FIG. 4A shows a Western blot carried out on preparations of membranes of oocytes injected with 10 (track 1) or 20 (track 2) ng of RNA encoding human Patched protein per oocyte. Track 3 corresponds to the control constituted by non-injected oocytes.

It is observed that the human Patched protein is expressed on the plasma membrane of the oocytes and that this expression is proportional to the quantity of RNA injected.

FIG. 4B shows the measurements of doxorubicin efflux on oocytes. The experiments being carried out on 4 lots of 10 control oocytes (non-injected) and oocytes injected with RNAs encoding human Patched protein, the average of the values obtained for the 4 lots is shown.

It is observed that the presence of human Patched protein on the plasma membrane of the oocytes causes an increase in the efflux of doxorubicin from the cell.

### EXAMPLE 5

### Binding of Doxorubicin to Human Patched Protein Expressed in Yeasts. (FIG. 5)

[symbol] : Control yeasts not expressing human Patched protein.
[symbol] : Yeasts expressing human Patched protein (preparation 1)
x: Yeasts expressing human Patched protein (preparation 2)
[symbol] : Yeasts expressing human Patched protein (preparation 3)

A rapid and significant quenching of the doxorubicin fluorescence is observed with the membranes containing human Patched protein corresponding to the binding of doxorubicin to the human Patched protein.

1. In vitro method for regulation of the intracellular concentration of anticancer agents characterized in that cells and a modulator of the activity of Patched protein are placed in contact in order to regulate the intracellular concentration of anticancer agents.

2. Method according to claim 1, characterized in that cells and a stimulator of the activity of transport of anticancer agents by Patched protein are placed in contact.

3. Method according to claim 1, characterized in that cells and an inhibitor of the activity of transport of anticancer agents by Patched protein are placed in contact.

4. A method for the regulation of the intracellular concentration of anticancer agents, comprising administering a modulator of the activity of Patched protein.

5. A method according to claim 4, wherein the modulator of the activity of Patched protein is an activator of the activity of Patched protein and wherein the medicament is intended to decrease the intracellular concentration of anticancer agents.

6. A method according to claim 4, wherein the modulator of the activity of Patched protein is an inhibitor of the activity of Patched protein and wherein the medicament is intended to increase the intracellular concentration of anticancer agents.

7. Method according to claim 1, wherein the anticancer agent is selected from the group consisting of antitumor antibiotics, alkylating agents, antimetabolites, plant alkaloids, topo-isomerase inhibitors and the mitotic spindle poisons.

8. Method according to claim 7, wherein the anticancer agent is an antitumour antibiotic.

9. Method according to claim 8, wherein the antitumour antibiotic is selected from the group consisting of bleomycin, daunorubicin, doxorubicin, epirubicin, hydroxyurea, idarubicin, mitomycin C and mitoxanthrone, preferably doxorubicin.

10. A method for improving the efficacy of an anticancer treatment, comprising administering a modulator of the activity of Patched protein.
